(19) United States
(12) Patent Application Publication
(10) Pub. No.: US 2026/0028597 A1
(43) Pub. Date: Jan. 29, 2026

(54) PURIFICATION OF BIOLOGICAL PARTICLES

(71) Applicant: Cytiva BioProcess R&D AB, Uppsala (SE)

(72) Inventors: Åsa HAGNER MCWHIRTER, Uppsala (SE); Nils Norrman, Uppsala (SE); Jean-Luc Maloisel, Uppsala (SE); Brigitta NÉMETH, Uppsala (SE)

(21) Appl. No.: 19/143,828

(22) PCT Filed: Dec. 11, 2023
(86) PCT No.: PCT/EP2023/085094

§ 371 Date: Jun. 26, 2025

(30) Foreign Application Priority Data
Dec. 27, 2022 (SE) 2251582-9

(51) Int. Cl. C12N 7/00; B01D 15/18; B01D 15/32; B01D 15/34; B01D 15/36; B01D 15/38; B01D 15/42; B01J 20/24; B01J 20/28; B01J 20/285; B01J 41/07; B01J 41/09; B01J 41/20

(52) U.S. Cl. CPC C12N 7/00; B01D 15/1871; B01D 15/327; B01D 15/34; B01D 15/363; B01D 15/388; B01D 15/424; B01J 20/24; B01J 20/28016; B01J 20/285; B01J 41/07; B01J 41/09; B01J 41/20; C12N 2740/00051

(57) ABSTRACT

A chromatography medium comprising porous beads having an inner porous core and an outer porous shell is used for chromatographic separation of enveloped or membranous biological particles from impurities such as contaminant DNA and/or protein. The core is capable of binding molecules via hydrophobic interactions; however, the pore size of the shell does not allow particles having a size of 20 nm and larger to permeate into the bead and interact with the core. The separation is performed at a pH of less than 7.4. The enveloped or membranous biological particles may have been subjected to a prior chromatographic capture step. When used for purification of enveloped virus particles the inventive process was found to yield a remarkably high rate of infectious virus particles.

### TECHNICAL FIELD

The present invention relates to a process for chromatographic purification of enveloped or membranous biological particles, and to use of a chromatography medium comprising porous beads having an inner porous core and an outer porous shell for purification of enveloped or membranous biological particles.

### BACKGROUND

Advanced therapy medicinal products (ATMPs) form an emerging area of great promise for the treatment or prophylaxis of severe diseases. It includes for instance gene and cell therapies aimed at restoring or replacing defective genes, as well as other therapies based recombinant nucleic acids, such as DNA and RNA vaccines.

To deliver a recombinant nucleic acid to a recipient cell, a vector is typically needed. Depending on the kind of therapy, the target cell type and the nucleic acid to be delivered, different vectors may be used. Commonly used vectors include enveloped virus particles, such a lentivirus (LV). Extracellular vesicles (EV), which are produced and released by cells, is another example of a vector with potential in cell and gene therapy.

The lentivirus (LV) is classed as retrovirus, and it has a single stranded RNA genome with a reverse transcriptase enzyme. Lentiviruses consist of a viral envelope with glycosylated proteins acting as ligands that has affinity to host cells receptors in the outer cell membrane surface. The virus effects transcription of the viral genetic material upon entering the cell. The viral genome consists of RNA sequences that code for specific proteins that facilitate the incorporation of the viral sequences into the host cell genome.

The virus infects the host cells by docking onto the host cell surface CD 4 glycoproteins. Next, the virus injects its material into the host cell cytoplasm where the reverse transcriptase enzyme generates reverse transcription of the viral RNA so that a viral DNA genome is produced, which is sent to the nucleus of the host cell and incorporated into the host cell genome. The host cell starts to transcribe the virus RNA and express viral proteins that form the capsid. The viral RNA and the viral proteins then assemble, and the new virions thus formed leave the host cell.

When an enveloped virus, like a lentivirus, is released from the host cell, the virus buds off from the host cell and thus has an outer lipid bilayer derived from the cell membrane containing viral glycoprotein. Inside the enveloped virus particle there is a protein capsid containing the viral genetic material. The envelope is critical for the infection of host cells, as it binds and fuses with host cell membrane.

For use in gene therapy, a virus is modified to act as a vector to insert beneficial genes into cells. The benefit using LV as viral vector is that it can penetrate the nuclear envelope in dividing as well as non-dividing cells, unlike other retroviruses that only penetrate cells undergoing mitosis. Many cell types in adult individuals do not divide, and LV might be the only option to transfer genetic material into such cells. Genetically modified LVs used in cell and gene therapy have proven to be promising candidate for curing diseases such as diabetes, prostate cancer, chronic granulomatous disease and vascular diseases. It is thus important to modify the virus genome so that it cannot replicate itself and that it is incorporated permanently in the cell genome. The transduction of human cells by genetically modified lentiviruses is most made ex vivo by transfection of human T-cells.

To produce a lentivirus, several plasmids are transfected into a so-called packaging cell line. One or more plasmids, generally referred to as packaging plasmids, encode the virion proteins, such as the capsid and the reverse transcriptase. Another plasmid contains the genetic material to be delivered by the vector. It is transcribed to produce the single-stranded RNA viral genome and is marked by the presence of the ψ (psi) sequence. This sequence is used to package the genome into the virion. To use lentivirus in gene therapy it is necessary to purify the virion from cell impurities like host cell proteins and DNA, and excess plasmids after transfection. Normally, the harvested host cells producing lentivirus are nuclease treated and the lentivirus is purified with several filtration techniques, such as normal flow microfiltration, ultrafiltration and diafiltration, to reduce the level of impurities to approved levels.

However, current downstream purification processes of lentivirus are often synonymous with low recovery (typically 10-20%) of infectious viruses, since lentiviruses are unstable and sensitive to shear forces, buffer components such as salt, and degrade quickly in room temperature. Time-consuming multistep processes are also not regarded as beneficial. Lentiviruses have been reported to be stable in a very narrow pH range, 7.0-7.4 (Kinetic Analyses of Stability of Simple and Complex Retroviral Vectors, F. Higashikawa et al., Virology 280, 124-131 (2001)), and conductivity window, <0.2M NaCl (Process development of lentiviral vector expression, purification and formulation for gene therapy applications, Doctoral thesis, Sara Nilsson, U C L, 2016), of processing solutions, which makes the downstream purification process challenging.

There is, hence, a need for improved or at least alternative methods of purifying encapsulated virus particles, like lentivirus particles, and other sensitive biological particles used as vehicles for cell or gene therapy or in the preparation of ATMPs.

### SUMMARY OF THE INVENTION

It is an object of the invention to overcome or at least partly alleviate drawbacks of the prior art. Accordingly, it is an object of the invention to provide an improved purification process by which enveloped or membranous biological particles, such as enveloped viruses, can be purified.

In one aspect, this and other objects are achieved by the use of a chromatography medium comprising porous beads having an inner porous core and an outer porous shell for chromatographic separation of enveloped or membranous biological particles from impurities such as contaminant DNA and/or protein. The core is capable of binding molecules via hydrophobic interactions, and the pore size of the shell does not allow particles having a size of 20 nm and larger to permeate into the bead and interact with the core. The separation is performed at a pH of less than 7.4, such as a pH in the range of from 6.0 to less than 7.4, such as from 6.5 to 7.2, such from 6.1 to 7.1. The separated enveloped or membranous biological particles are typically obtained in the flow-through. The enveloped or membranous biological particles may have been subjected to a prior chromatographic capture step resulting in a partially purified product containing enveloped or membranous biological particles and contaminant DNA, before being applied to said chromatography medium comprising porous beads.

Performing the chromatographic separation using a porous shell/bead chromatography medium as described herein (and which herein is alternatingly referred to as a polishing step), at the indicated pH value, was found to yield a surprisingly high rate of infectious virus particles. It is contemplated that such a chromatography step as such may provide 70-100% infectious recovery (relative to the input material), while also being very effective for removal of small sized impurities, such as residual host cell protein and DNA. The high infectious recovery obtained when used for purification of enveloped lentivirus particles is also an indication that the process is mild enough to be suitable for purification of similarly sensitive, enveloped or membranous biological particles, such as other enveloped viruses, or extracellular vesicles.

In another aspect, the invention provides a process of purifying enveloped or membranous biological particles from a feed, the process comprising:
a) adding a feed comprising enveloped or membranous biological particles and one or more impurities to a first chromatography medium at a pH of 6-10, preferably 6-8, the first chromatography medium comprising a support material that is functionalized with a ligand that captures said enveloped or membranous biological particles,
b) eluting the enveloped or membranous biological particles from the first chromatography medium in at least one eluate fraction containing enveloped or membranous biological particles,
c) adjusting, if needed, the pH of the eluate fraction of step b) to a pH of less than 7.4,
d) adding the eluate fraction, at a pH in the range of from 6.0 to less than 7.4, to a second chromatography medium comprising porous beads having an inner porous core and an outer porous shell, wherein the core is capable of binding molecules via hydrophobic interactions, and wherein the pore size of the shell prevents particles having a size of 20 nm and larger from contacting the core, and
e) obtaining at least one flow-through fraction containing purified enveloped or membranous biological particles from the chromatography medium of step d.

The present process is advantageous for purification of sensitive biological particles in several aspects. As demonstrated herein, performing the step d at a pH below 7.4 allows a remarkable increase of recovered infectious virus particles as compared to a pH of 7.4. For example, for enveloped virus particles, the present process (the totality of steps a-e) may enable an infectious recovery of at least 30%, such as at least 50%, in relation to the content of infectious virus particles in an untreated feed. This represents a substantial improvement over currently available methods, which typically yield an infectious recovery of 10-20%. Using a feed that has been clarified prior to step a, the present process steps a-e may result in an infectious recovery of at least 50%, such as at least 60%, such as 70%, in relation to the clarified feed.

Looking at the individual chromatography steps separately, the first capture (steps a-b) may provide an infectious recovery of enveloped virus particles of at least 60%, such as at least 70% in relation to the feed. The polishing using a porous shell/bead resin (step d-e) as such may independently yield an infectious recovery of at least 70%, and up to 100%, relative to the content of infectious virus particles provided by the preceding step.

Furthermore, combining chromatographic capture with a polishing step as described herein, in which the purified enveloped or membranous biological particles are obtained in the flow-through, offers a short process time, which is considered beneficial to stability. Furthermore, it is also believed that the biological particles experience relatively low shear forces during the flow-through step, which also contributes to maintaining integrity and/or infectious properties.

The enveloped or membranous biological particles may be selected from enveloped virus particles and extracellular vesicles, such as exosomes. Enveloped virus particles may for instance be lentivirus particles.

The term “virus particle” is herein used to denote a complete infectious virus particle. It includes a core, comprising the genome of the virus (i.e., the viral genome), either in the form of ribonucleic acid (RNA) or deoxyribonucleic acid (DNA), and the core is surrounded by a morphologically defined shell. The shell is called a capsid. The capsid and the enclosed viral genome together constitute the so-called nucleocapsid. The nucleocapsid of enveloped viruses is surrounded by a lipoprotein bilayer envelope. In the field of bioprocessing, for the purpose of producing viral vectors for various applications such as therapy, the genome of a virus particle is modified to include a genetic insert, comprising genetic material of interest.

The term “impurities” is herein intended to mean any molecule or substance which is present in the liquid sample, and which is not the desired target entity. In the context of the present invention, “impurities” includes primarily host cell proteins (HCP), and host cell DNA. However, the term “impurities” generally also includes aggregates, such as aggregates of the target entity, and fragments of the target entity. In the context of the present invention, the target entity is the enveloped or membranous biological particle.

The expression “functionalized with a ligand” used herein means that a ligand is chemically coupled, optionally covalently, to the entity that is functionalized according to conventional methods. The coupling may involve use of a linker, a spacer or a surface extender. In this context, “ligand” is a molecule that is capable of binding a given analyte or binding partner. The binding may be specific, as in the case of affinity ligands, which bind a particular target entity, or non-specific, as in the case of hydrophobic interaction ligands or ion exchange ligands which are capable of binding any entity having the required properties (e.g., electric charge). It is to be understood that “a ligand” is intended to mean a ligand species and that the singular form of the term may encompass a large number of individual ligands.

The support material of the first chromatography medium may be selected from monoliths, membranes, porous beads, non-porous beads, magnetic beads, or expanded bed media.

The ligand of the first chromatography medium that captures said enveloped or membranous biological particles may be selected from affinity ligands and anion exchange ligands. The ligand may be an anion exchange ligand comprising a diamine functionality generating at least one weak anion exchange group to an ionic capacity of 10-500 μmol/mL. The anion exchange group is positively charged or partially positively charged at a pH of 6-10.

A suitable anion exchange ligand may be described by the formula (I):

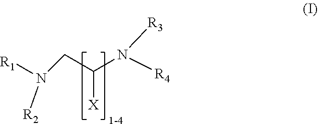

wherein X, for each occurrence independently, is selected from H, OH or a C₁₋₃ group, and R₁, R₂, R₃ and R₄ are independently selected from H, and a C₁₋₃ group,
wherein a C₃ group is straight or branched,
wherein a C₁₋₃ group comprises groups independently selected from OH, O—C₁₋₂, S—C₁₋₂, NH, NHR, NR₂, wherein R is selected from H and a C₁₋₃ group.

For example, a suitable AIEX ligand may be selected from N,N,N′-triethylethylenediamine, diethylenetriamine, N,N′-dimethylethylenediamine, N-methylethylenediamine, 1,3-diaminopropane, 1,3-diamino-2-hydroxypropane, 2-methyl-1,3-propanediamine and N,N-diethylethylenediamine. N,N-diethylethylenediamine may be preferred.

In the present process, the eluting of step b) may be provided by contacting the first chromatography medium with an elution buffer having a salt concentration of at most 0.65 M. Elution may be provided using an increasing salt concentration.

The eluate fraction may be added to the chromatography medium in step d) at a pH of from 6.5 to 7.2, such as from 6.5 to 7.1, or from 6.8 to 7.1.

In embodiments, the at least one flow-through fraction containing purified enveloped or membranous biological particles obtained in step e) contains no detectable DNA.

In the present process or use, the porosity or pore size of the shell may be such that it does not allow molecules larger than 700 kDa to contact the core. For the purpose of purifying relatively small enveloped or membranous biological particles, the porosity or pore size of the shell may be such that it does not allow molecules larger than 400 kDa to contact the core. In terms of particles sizes, the porosity or pore size of the shell may exclude particles having a size of 20 nm and above from contacting the core, or particles having a size of 30 nm and above, or particles having a size of 60 nm and above, such as 100 nm and above.

The porous beads may comprise a hydrophilic polymer, such as a polysaccharide, such as agarose. Preferably the core and the shell may comprise agarose.

The porous core may be functionalized with a hydrophobic interaction ligand. The hydrophobic interaction ligand may comprise an aliphatic or aromatic C₄ to C₁₆ hydrocarbon, such as an aliphatic C₄ to C₁₆ hydrocarbon. The hydrophobic interaction ligand is a C₄ to C₁₆ alkylamine, such as octylamine.

In the present process or use, the residence time of the enveloped or membranous biological particles in the chromatography medium comprising porous beads having an inner porous core and an outer porous shell may between 0.5 and 10 minutes. The flow rate may be from 0.1 mL/min to 3 mL/min.

Optionally, the feed may have been subjected to nuclease treatment prior to the present chromatography steps. In the present process, a nuclease treatment may be carried out prior to step a).

Preferred aspects of the present disclosure are described below in the detailed description and in the dependent claims. It is noted that the invention relates to all possible combinations of features recited in the claims.

### BRIEF DESCRIPTION OF THE DRAWINGS

This and other aspects of the present invention will now be described in more detail, with reference to the appended drawings, in which:

FIG. 1 is a flow chart outlining the steps of the process according to the invention.

FIG. 2 schematically illustrates in cross-section an exemplary capture chromatography device used for purification of enveloped or membranous biological particles.

FIG. 3 schematically illustrates in cross-section an exemplary chromatography bead as used in the present invention, for purification e.g. of lentivirus (LV).

FIG. 4 is a chromatogram for capture of lentivirus using Capto™ DEAE anion exchange performed as described in the experimental section.

FIG. 5 is a graph showing % total (p24) and infectious lentivirus recovery and impurity (DNA, HCP) reduction after Capto™ DEAE capture performed as described in the experimental section. On the x-axis, “FT” denotes flow-through, and E1-5 represent eluate fractions 1-5. A high level of infectious lentivirus particles was obtained, especially in fraction E3, and co-eluted with DNA.

FIG. 6 is a graph showing total (p24) and infectious recovery, respectively, of lentivirus purified using Capto™ Core 700 as set out in the experimental section, at different pH. A pH of 7.0 resulted in a significant improvement compared to pH 8.0 and pH 7.4.

As illustrated in the figures, some features may be exaggerated for illustrative purposes and, thus, are provided to illustrate the general structures of embodiments of the present invention.

### DETAILED DESCRIPTION

The present invention is useful for purifying enveloped or membranous biological particles, which typically are sensitive to shear forces, salt, pH and detergents. Enveloped or membranous biological particles may be of natural or synthetic origin, and may be obtained from a biological sample, or produced in vivo by recombinant expression in prokaryotic or eukaryotic cells, or produced in vitro. The envelope of an enveloped biological particle may comprise a lipid bilayer. The membrane of a membranous biological particle may comprise a lipid bilayer.

Enveloped biological particles may be enveloped viruses. Examples of enveloped viruses are DNA viruses, such as herpesvirus, oxvirus, hepadnavirus and asfarviridae; RNA viruses, such as flavivirus, alphavirus, togavirus, coronavirus, hepatitis D, orthomyxovirus, paramyxovirus, rhabdovirus, bunyavirus and filovirus; and retroviruses such as lentiviruses. Membranous biological particles may be extracellular vesicles, such as exosomes. Additionally, virus-like particles (VLPs) are included enveloped or membranous biological particles.

The enveloped or membranous biological particles may contain a genetic cargo, intended e.g. for gene therapy. The genetic cargo may be one or more exogenous nucleic acid sequences contained together with the native nucleic acid content of the particle. In the case of an enveloped virus particle, the viral genome may contain an exogenous DNA or RNA insert. An enveloped or membranous biological particle such as a virus particle containing an exogenous genetic insert may be recombinantly produced according to known methods. The presence of a large genetic insert (exogenous DNA or RNA) may render a virus particle more sensitive and unstable.

The present invention provides a mild yet effective purification process, which is useful for purification of sensitive enveloped or membranous biological particles. As demonstrated herein, the present invention is particularly useful for purifying an enveloped retrovirus, such as a lentivirus, at very high infectious recovery rate.

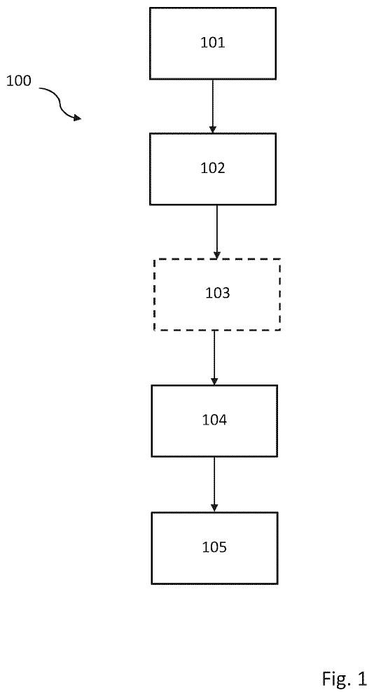

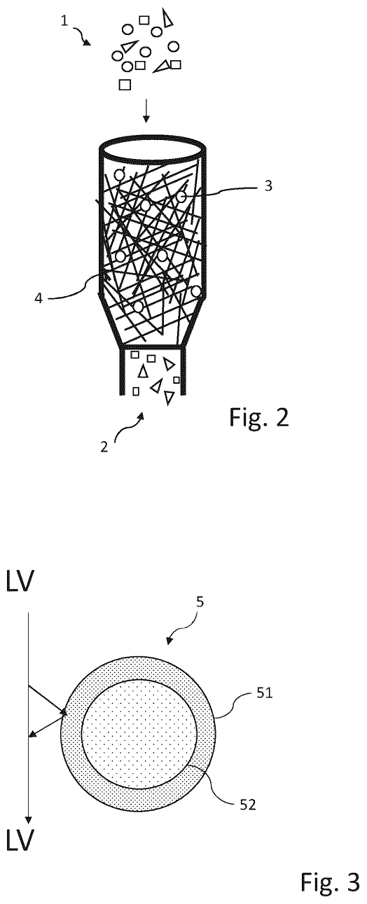

FIG. 1 schematically illustrates a process 100 for purifying enveloped or membranous biological particles, and FIG. 2 illustrates the principle of the AIEX chromatography employed.

The enveloped or membranous biological particles 3 are present in a feed 1 together with one or more impurities 2. For example, a feed comprising the enveloped virus particles such as lentivirus particles or exosomes may be produced by a cell line such as for example HEK 293 cells (human embryonic kidney cells). The feed may have been clarified or otherwise pretreated according to known methods before being applied to the first chromatography medium. In a clarified harvest, the amount of solids has been reduced and the one or more impurities may be soluble impurities such as host cell proteins (HCP) and DNA. For example, the feed may be subjected to treatment with a nuclease in order to partially degrade and reduce the size of contaminant polynucleotides. In the case of purification of an RNA virus, treatment with a DNAse may be applied.

In a first step 101 of the process 100 the feed 1 is added to a first chromatography medium 4, at a pH of 6-10, for capture of the enveloped or membranous biological particles. For the purpose of capturing enveloped or membranous biological particles, the chromatography medium 4 comprises a support material that is functionalized a ligand capable of reversibly binding the target enveloped or membranous biological particles.

The ligand can be an affinity ligand capable of binding the relevant enveloped or membranous biological particle. Alternatively, the ligand may be an anion exchange (AIEX) ligand, and preferably a weak AIEX ligand.

The feed 1 is typically loaded onto the first chromatography medium 4 using a loading buffer having a pH in the range of 6-10, such as 6-8. Prior to loading the medium 4 may be equilibrated using the loading buffer or another buffer having the appropriate pH of 6-10, such as 6-8. Under loading conditions, the enveloped or membranous biological particles bind to the ligands, whereas at least some impurities 2, in particular host cell proteins, are washed through the column. The feed can be loaded at a flow rate of from 1 to 10 mL/min.

After loading of the feed and optional additional washing, follows a step 102 of eluting the enveloped or membranous biological particles. The elution may be achieved by altering the ionic strength. For instance, an elution buffer providing an increased concentration of a salt may be used.

The salt may comprise (i) an anion selected from a group consisting of CO₃²⁻, SO₄²⁻, S₂O₃²⁻, H₂PO₄⁻, HPO₄²⁻, acetate-, citrate-, and Cl⁻, and (ii) a cation selected from a group consisting of NH₄⁺, K⁺, Na⁺, and Li⁺. For example, the salt may comprise sodium acetate (NaOAc) or sodium chloride (NaCl). However, it is to be understood that other salts consisting of a combination an anion as listed under (i) and a cation as listed under (ii) may alternatively be used to elute the capsids. Non-limiting examples of such other salts are LiCl, KCl, or other equivalent metal salt suitable to use for salt elution, as is well known in the art. Non-limiting examples of suitable concentrations of NaCl include from about 5 mM to about 1M, such as about 5, 10, 20, 30, 40, 50, 100, 150, 200, 250, 300, 350, 400, 450, 500, 600, 700, 800, 900, or 1000 mM. Non-limiting examples of suitable concentrations of NaOAc include from about 5 mM to about 500 mM, such as about 5, 10, 20, 30, 40, 50, 60, 70, 80, 90, 100, 150, 200, 250, 300, 350, 400, 450, or 500 mM.

During elution (step 102), the salt concentration may be increased stepwise or continuously. A linear gradient of NaCl ranging from about 100, such as 130, to about 650 mM may be used. Such a relatively low salt concentration is beneficial as enveloped viruses and exosomes have a reduced stability at higher conductivities, and especially so at concentrations higher than 0.65 M. Optionally the salt concentration during elution may be at most 450 mM.

Elution can be carried out at a flow rate of 1 to 10 mL/min. The flow rate may be the same as during loading.

Typically, elution enveloped or membranous biological particles from the first chromatography medium having a support material being a convection-based fibrous substrate, such as a fibrous non-woven polymer matrix, can be made at flowrates down to a few seconds residence time, i.e. 60 MV/min, and if required up to several minutes residence time (max 6 min) 0.2 MV/min. Optimal residence time for such fibrous support materials is 5-20 MV/min. For resins, typical residence times are 1-8 minutes and a sufficient residence time is often obtained after 4 minutes.

Optionally, the elution buffer may contain arginine, or another additive that is conventionally used for elution in affinity or anion exchange chromatography, provided that the additive does not interfere with the ability of second chromatography medium to bind impurities and allow the enveloped or membranous biological particles to flow through.

At least one fraction of eluted enveloped or membranous biological particles is thus provided. Optionally, more than one fraction may be corrected and optionally pooled, before being subjected to the next step of the process.

In a method of purifying an enveloped virus such as lentivirus, the eluted fraction(s) containing the virus typically contain a high level of recovered infectious virus particles. For example, an infectious recovery of at least 50%, such as at least 60%, at least 70%, or at least 80%, may be achieved after the step 102.

In the elution step, direct dilution to decrease conductivity in a buffer containing sucrose could stabilize the virus and thereby improve the recovery.

The first chromatography medium may be functionalized with ligands to an ionic capacity (number of charged functional groups per ml medium (μmol/ml)) of 10-500 μmol/mL, or in the range of 50-300 μmol/mL or 100-300 μmol/mL. The ligand may have a diamine functionality generating at least one weak anion exchange group ionic capacity (number of charged functional groups per ml medium (μmol/ml)) of 10-500 μmol/mL, or in the range of 50-300 μmol/mL or 100-300 μmol/mL. A weak anion exchange group may comprise multimodal weak anion-exchange groups, i.e. the anion-exchange group provides at least two different, but co-operative, sites which interact with the compound to be bound (i.e. the enveloped or membranous biological particles). For example, one of these sites may give an attractive type of charge-charge interaction between the ligand and the binding target. The other site may contribute to the binding by introducing a second local charge or by increasing the local amount of solvating water, which is impacting the binding capacity.

A weak anion exchange group may be positively charged or partially positively charged at a pH of 6-10. Such positively charged or partially positively charged weak anion exchange group may attract an enveloped or membranous biological particle that is negatively charged at neutral pH, such as lentivirus particles. A weak anion exchange group may be positively charged or partially positively charged at a pH of 6-10 or, 6-9.5, 6-9 or 6-8.

Weak ionic exchange groups means that there is a gradient according to the pH from fully charged to not charged, having at PI a neutral charge (same amount of + and −). Strong anionic exchange groups, based on a quaternary amine, are on the contrary always charged. Almost all other anionic exchange groups, not based on a quaternary amine, are weak, i.e. the charge varies (and can be zero) within a reasonable range of pH used (such as e.g. pH 2-11).

The ligand or a portion of the ligand may be described by the formula (I):

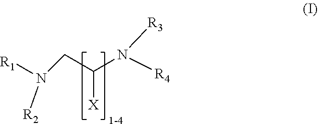

wherein X, for each occurrence independently, is selected from H, OH or a C₁₋₃ group, and
R₁, R₂, R₃ and R₄ are independently selected from H, and a C₁₋₃ group,
wherein a C₃ group is straight or branched,
wherein a C₁₋₃ group comprises groups independently selected from OH, O—C₁₋₂, S—C₁₋₂, NH, NHR, NR₂,
wherein R is selected from H and a C₁₋₃ group.

The ligand or a portion of the ligand may be described by the formula (I) above. This means that the diamine ligand can constitute part of a larger structure such as a polymer. The ligand or a portion of the ligand can be part of larger structures generated for example by the reaction of the solid support with lower molecular weight amine chemical containing leaving groups such as 2-chloro-N,N-diethylethylamine (DEAE), 2-chloro-N,N-diethylethylamine, 2-chloroethylamine, 3-chloropropylamine, 2-chloro-N,N-dimethylethylamine, 3-chloro-N-methylpropan-1-amine. In the ligand or the portion of the ligand comprising a diamine functionality generating at least one weak anion exchange group described by the formula above, the two amines may be separated by 2-4 carbon atoms, each amine group may be substituted by two R groups, which may be chosen from H and alkyl groups C₁₋₄ and that can be branched and/or also substituted by other groups such as hydroxyl, amines, ether and thio ether, these later being restricted to 3-8 atoms.

The ligand described by the formula above may be selected from N,N,N′-triethylethylenediamine, diethylenetriamine, N,N′-dimethylethylenediamine, N-methylethylenediamine, 1,3-diaminopropane, 1,3-diamino-2-hydroxypropane, 2-methyl-1,3-propanediamine and N,N-diethylethylenediamine.

The support material of the chromatography medium 4 may be selected from monoliths, membranes, filters, porous beads, non-porous beads, magnetic beads, or expanded bed media.

Beads of different bead sizes may be used, such as beads having a diameter of 1-120 μm, or 10-120 μm. The beads may comprise a polysaccharide, such as agarose, which may be cross-linked. The beads may be of homogeneous porosity.

Monoliths are single pieces of porous materials characterized by a highly interconnected network of channels with diameters in the range of 10-4000 nm.

Membrane materials may be inorganic-organic (e.g., an alkoxysilane coated on glass fiber), alumina membranes and organic materials (i.e., cellulose and its derivatives, regenerated cellulose, nylon, polyethersulfone, polypropylene, polyvinylidene, etc).

The support material may be a woven material. Alternatively, the support material may be a non-woven material, for example comprising fibers, such as from cellulose, having a mean flow pore size of 0.1-2.0 μm. Mean flow pore (MFP) size is an indicator of material flow characteristics, and is measured by capillary flow porometry, based on the displacement of a wetting liquid with a known surface tension from the sample pores by applying a gas at increasing pressure. The higher the MFP size, the larger the flow of liquid through the material at a given pressure. The mean flow pore size is calculated from the point at which 50% of the flow goes through a sample. Mean flow pore size thus corresponds to the pore size calculated at the pressure where the wet curve and the half-dry curve meet. In an alternative definition, the mean flow pore size of a support material may be seen as an effective pore size defined as the size of the largest sphere that is able to pass through the pore.

The mean flow pore size of the support material may be 0.1-1.8 μm, 0.1-1.6 μm, 0.1-1.4 μm, 0.1-1.2 μm, 0.1-1.0 μm, 0.1-0.8 μm, 0.1-0.6 μm, 0.1-0.4 μm, 0.1-0.2 μm, 0.2-2.0 μm, 0.4-2.0 μm, 0.6-2.0 μm, 0.8-2.0μ, 1.0-2.0 μm, 1.2-2.0 μm, 1.4-2.0 μm, 1.6-2.0 μm, 1.8-2.0 μm, or 0.5-1.5 μm.

The chromatography support material could comprise a convection-based chromatography matrix. Said convection-based chromatography matrix may be a fibrous substrate. Said fibrous substrate may be based on electrospun polymeric fibers or cellulose fibers, optionally non-woven fibers. The fibrous substrate may thus be a fibrous non-woven polymer matrix. The fibers comprised in said fibrous substrate have a cross-sectional diameter of 10-1000 nm, such as 200-800 nm, 200-400 nm or 300-400 nm. Such a fibrous substrate can be found in a HiTrap Fibro unit from Cytiva, Sweden. Typically, elution of enveloped or membranous biological particles from a chromatography medium having a support material being a convection-based fibrous substrate, such as a fibrous non-woven polymer matrix, can be made at flowrates down to a few seconds residence time, i.e. 60 MV/min, and if required up to several minutes residence time (max 6 min) 0.2 MV/min. Optimal residence time for such fibrous support materials is 5-20 MV/min. For resins, typical residence times are 1-8 min and a sufficient residence time is often obtained after 4 min.

The ligand of the first chromatography medium 4 may be connected to the support material through an extender group, which may be selected from polysaccharide structures and polymeric structures. The extender group may be e.g. dextran, acrylamides or polyglycidol. If dextran is used as extender it may have a molecular weight in the range of from 5 000 to 2M Dalton. The extender group, if present, may be selected with regard to the type of support material and ligand that is to be immobilized/connected to the support material.

The eluted fraction obtained in step 102 which contains enveloped or membranous biological particles typically also still contains one or more types of impurities, although significant amount of impurities may have been removed during the capture chromatography. Examples of impurities that may bind and elute together with the enveloped or membranous biological particles include host cell DNA (hcDNA). For example, the eluate may contain at least 50% of the hcDNA present in the feed. DNA is negatively charged and binds strongly to anion exchangers, and longer DNA sequences may bind stronger than shorter DNA fragments. It has previously been found that the higher the elution conductivity for a virus particle, the higher the risk of coelution of the virus particle with DNA. Therefore, lower elution conductivity may be preferred to reduce hcDNA in the target elution fraction.

The eluate fraction obtained in the step 102, which contains enveloped or membranous biological particles and typically also some remaining impurities, such as host cell DNA (preferably fragmented by prior nuclease treatment), is added to a second chromatography medium comprising porous beads having a core-shell structure, wherein the core is functionalized with a hydrophobic interaction ligand. In FIG. 1, this is represented by step 104. The impurities are retained by the porous beads, and the enveloped or membranous biological particles are obtained 105 in the flow-through.

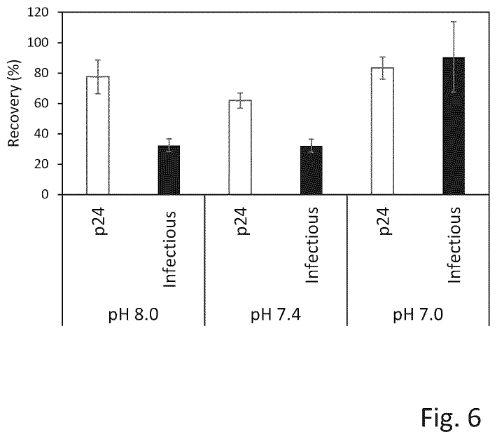

The chromatography step 104 is performed at a pH in the range of from 6.0 to less than 7.4, such as from 6.5 to 7.2, such as 6.5 to 7.1, or from 6.8 to 7.1. Therefore, if the eluate fraction obtained in step 102 has a pH outside of the desired range, for example a pH of 7.4 or higher, the pH is suitably adjusted in step 103 prior to addition to the second chromatography medium. The pH adjusting step 103 may be realized by diluting the eluate fraction with a buffer having a lower (if lowering is desired) until the desired pH is reached in the diluted eluate fraction. Of course, should a pH increase be desired, e.g. from a value below 7.0, a dilution buffer having a pH higher than the eluate fraction can be used. The buffer used for dilution may be the same buffer as used as a loading buffer in the step 104. As illustrated in FIG. 6, comparative examples using pH values of 7.4 and 8, respectively, for this step resulted in a low infectious recovery of lentivirus particles, whereas performing step 104 at a pH of 7.0 resulted in a most remarkable increase in infectious recovery. A satisfactory recovery is believed to occur also slightly above 7.0. Not wishing to be bound by any particular theory, it is believed that enveloped or membranous biological particles may be slightly more tolerant to pH below 7.0 as compared to values above 7.0.

For clarity, if the pH of the eluate fraction is within the range 6.0 to less than 7.4, the step of adjusting 103 the pH is optional. However as indicated above, a pH of higher than 6.0, such as at least 6.5, or at least 6.8, may still be preferred. Hence, in such cases where the pH is at least 6.0 but lower than a preferred value, the pH may be suitably adjusted in step 103. Similarly, when the pH is below 7.4 but higher than a preferred value, the pH may be suitably adjusted by step 103.

FIG. 3 illustrates schematically a chromatography bead 5 of the second chromatography medium of step 104. The bead 5 is a porous bead comprising a porous core 52 surrounded by a porous shell 51. The porosity of the core and the shell may be the same or different. However, at least the porosity of the shell 51 prevents large entities, such as enveloped or membranous biological particles (represented by “LV” in FIG. 3), from permeating through the shell to contact the core.

The shell 51 is typically hydrophilic. Thus, the surface of the porous bead 5 that is accessible to large entities, such as enveloped or membranous biological particles, is hydrophilic and does not irreversibly adsorb or denature proteins. The shell may be formed of a hydrophilic material that exposes a plurality of polar groups, for instance comprising oxygen and/or nitrogen atoms. Examples of such polar groups are hydroxyl, amino, carboxy, sulphonate (S and SP ligands) ester, ether of lower alkyls (such as (—CH₂CH₂O—)ₙH where n is an integer 2, 3, 4 and higher).

The core 52 strongly binds biomolecules such as proteins and DNA through hydrophobic interactions. The core may be hydrophobic. Preferably, the hydrophobic core 52 is hydrophilic per se and is based on a hydrophilic material, such as a hydrophilic polymer, and is functionalized with a hydrophobic interaction ligand to provide the desired hydrophobicity. However, alternatively, the core may be hydrophobic per se, based on a hydrophobic polymer. For example, styrene/ethylstyrene/DVB, vinylethers and acrylates containing hydrophobic substituents as well as fluoroalkane-containing polymers are contemplated.

Hydrophobic interaction ligands may comprise aliphatic hydrocarbons, such as C1-C30 alkyl, preferably C4-C16 alkyl, and/or aromatic hydrocarbons, such as phenyl, antracene, naphtalene. Preferably, the ligand density may be more than 90 μmole/ml of core entity, which represents a ligand density higher than the level normally used for HIC resins. Advantageously, the hydrophobicity provided by the ligand may be such that proteins can interact or be adsorbed both at very low and high ionic strengths.

A hydrophilic polymer on which the shell and optionally the core may be based is a polysaccharide, such as agarose. For example, the core and the shell may both comprise cross-linked agarose. The porous core-shell beads may be produced as described in WO 2009/131526. In particular, the core and the shell may be made of agarose, and the core may be functionalized with hydrocarbon interaction ligands comprising 4-16 carbons, preferably octyl ligands. Useful chromatography media are available under the trade names Capto™ Core 400 and Capto™ Core 700, respectively, available from Cytiva, Sweden.

The shell covers or surrounds the core. Due to the limited porosity of the shell 51, only entities, such as molecules, small enough to penetrate the shell will be able to interact with the hydrophobic core. Hence, the porous beads 5 have a size exclusion property. In the present context, impurities such as remaining host cell proteins and DNA (preferably fragmented DNA), can enter the porous bead and bind, whereas the target enveloped or membranous biological particles cannot enter, but are obtained in the flow-through fraction.

The hydrophobic core is capable of strongly binding any impurities that permeate into the bead 5. Thus, under the present processing conditions, the impurities are not released from the core, but remains bound thereto.

The porosity of the shell may be defined as not allowing particles having a size (diameter) of 20 nm to enter the bead, such that particles of this size and larger are not retained in the chromatography medium, but allowed to flow through it, whereas smaller particles can enter the bead to be adsorbed or bound by the hydrophobic core. For example, the shell may have a porosity small enough to prevent particles having a size of at least 30 nm, such as 80 nm and larger.

Enveloped viruses generally have sizes ranging from 20 nm and up to 300 nm, depending on the type of virus. Lentiviruses may have a size in the range of 80-120 nm, often 100-120 nm. Enveloped viruses may be larger than non-enveloped viruses such as an adenovirus, which is packaged only in a capsid. Enveloped viruses are often larger than adeno-associated viruses, which are typically about 25 nm in size. Extracellular vesicles such as exosomes may have a size in the range of from 30 to 180 nm.

In alternative definitions, the porosity of the shell may be described as excluding molecules having a molecular weight of 400 kDa and higher, whereas molecules having a molecular weight below 400 kDa can enter the bead and interact with the hydrophobic core. In embodiments, the porosity of the shell may exclude molecules having a molecular weight of 700 kDa and higher, or a molecular weight of 1,000 kDa and higher, or a molecular weight of 2,000 kDa and higher. In contrast, having a molecular weight below the respective limit can enter the bead and interact with the hydrophobic core. A low cutoff value such as 400 kDa may be useful for purifying relatively small particles, such as having a size in the range of 20-100 nm. Higher cutoff values, such as 1,000 or 2,000 kDa may be used when the target particles to be purified is large, e.g. 150-300 nm.

The shell 51 may be inert, or non-functionalized. For example, the shell is not functionalized with the HIC ligand of core, nor any other HIC ligand. The porous shell may be hydrophilic.

The chromatography step 104 can be performed at a flow rate of from 0.1 mL/min to 3 mL/min, preferably 0.3 mL/min to 2 mL/min, such as 0.3 mL/min to 1.4 mL/min for a 1 mL column. The flow rate for step 104 may be lower than the flow rate used in the previous steps 101 and/or 102.

The residence time of the sample (eluate obtained in step 102 and optionally diluted) in the second chromatography medium in step 104 may be from 0.5 to 10 min, such as from 0.5 to 3.5 min. For example, the retention time may be 1 minute or less, such as from 0.5 to 0.7 min. Longer residence times (slower flow rates), may provide higher purity, as impurities will have more time to permeate into the porous beads and interact with the core 52. Shorter retention times (faster flow rates) may favor process efficiency and economics. Also, for sensitive biological particles like enveloped viruses, a short process time may be preferred to minimize exposure to conditions (e.g., room temperature) that may have a negative impact on virus stability.

Advantageously, the process described herein may be performed on large sample volume in relation to the volume of the chromatography media. Hence, a relatively large feed volume can be used in relation to the volume of the first chromatography medium, which may have a high dynamic binding capacity, especially when the support material is a fibrous material. Further, a relatively large volume of sample (the eluate obtained from the capture chromatography steps, optionally diluted to adjust the pH as described above), can be applied in step 104 to the second chromatography medium comprising porous beads 5. For example, the sample volume applied may represent 15-30 column volumes (CV), such as 25-30 CV, wherein the column volume is the volume of the chromatography medium comprising the porous beads 5.

As demonstrated herein, performing the step d at a pH below 7.4 can provide a remarkable increase of recovered infectious virus particles as compared to a pH of 7.4. For example, for enveloped virus particles, the present process 100 may enable an infectious recovery of at least 30%, such as at least 50%, in relation to the content of infectious virus particles in an untreated feed. This represents a substantial improvement over currently available methods, which typically yield an infectious recovery of 10-20%.

Pretreatment, such as clarification, of a cell culture harvest to provide a clarified feed to be used in step 101, may result in minor loss of infectious virus. In general, the infectious recovery after clarification may be at least 80%. Using a feed that has been clarified prior to step 101, the present process 100 may result in an infectious recovery of at least 50%, such as at least 60%, such as 70%.

Looking at the individual chromatography steps separately, the first capture (steps 101 and 102) may provide an infectious recovery of enveloped virus particles of at least 60%, such as at least 70% in relation to the feed. The polishing using a porous shell/bead resin (step 104 and 105) as such may independently yield an infectious recovery of at least 70%, and up to 100%, relative to the content of infectious virus particles provided by the preceding step.

Generally, addition of a stabilizer such as sucrose may be beneficial to the stability of the enveloped or membranous biological particles. The stabilizer may be added in all mobile phases and in formulation solutions used in the present process.

While the invention is described herein with respect to exemplary embodiments, the skilled person will appreciate that the invention is not limited to these. In the claims, any reference signs placed between parentheses shall not be construed as limiting the claim.

Use of the verb “to comprise” and its conjugations does not exclude the presence of elements or steps other than those stated. The article “a” or “an” preceding an element does not exclude the presence of a plurality of such elements. The mere fact that certain measures are recited in mutually different dependent claims does not indicate that a combination of these measures cannot be used to advantage.

### EXPERIMENTAL

### Determination of Ionic Capacity

The dynamic small ion binding capacity (DBC) of a chromatography material functionalized with AIEX ligands, such as diethylethanolamine (DEAE), quaternary amine (Q), or N,N-diethylethylenediamine (DAX), is described below with reference to a nanofibrous chromatography material (Fibro™ membrane adsorber, Cytiva, Sweden), and should be generally applicable for any AIEX ligand. The method uses a conductometric titration where added HCl protonates deprotonated weak ALEX ligands or neutralizes and displaces OH-bound to strong AIEX ligands. In contrast to protein DBC methods, the conductivity signal, rather than a UV signal, of the permeate is monitored in the ÄKTA system.

25 mm diameter membrane discs were attached in a membrane holder device suited to allow chromatography of the membranes, and run on an ÄKTA Explorer 10 system (Cytiva, Sweden) equipped with sample pump. The method consists of the following steps:
1. Rinsing of ÄKTA system and membrane (loaded in PEEK device).
2. Loading of membrane with excess NaOH
3. Rinsing of membrane with MQ water
4. Rinsing HCl solution in bypass
5. Loading membrane with HCl and monitor conductivity breakthrough
6. Reloading membrane with excess NaOH (optional, prep for normalization by weight)
7. Rinsing of membrane with MQ water

Each membrane batch is analyzed with triplicate discs. Normalization is done by disc volume and optionally also through disc dry weight.

### Determination of Mean Flow Pore Size

The mean flow pore size of a porous material can be measured with capillary flow analysis using commercially available equipment. In this example, the equipment used is a POROLUX™ 100 porometer (IB-FT GmbH, Berlin, Germany) according to the manufacturer's manual and methodology as given in Table 1.

TABLE 1
Capillary flow porometry

| Porometer | Porometer model | Porolux 100 | |
|---|---|---|---|
| specifications | Porometer algorithm | Pressure Scan | |
| | Measurable pore size | 90 nm-500 μm | |
| | Pressure range | 0-7 | bar |
| | Flow rate | up to 100 liters per minute | |
| | First Bubble Point | Calculated - according | |
| | | to ASTM F 316-03 | |
| Settings | Calculated bubble point | First Flow | |
| | method used | | |
| | Slope of pressure increase | 6.25 mbar/s (160 s/bar) | |
| | Number of data points for | 50 | |
| | wet curve | | |
| | Number of data points for | 25 | |
| | dry curve | | |
| | Test Gas used | Nitrogen | |
| | Fluid angle (°) | 0 (Default) | |
| | Shape factor name | 1 (Default) | |
| | Shape factor | 1 (Default) | |
| | Temperature | 21° | C. |
| Wetting fluid | Wetting Fluid | Porefil (Perfluoropolyether) | |
| | Surface Tension | 16.45 ± 0.02 | mN/m |
| | Tensiometer method for | 22° C., 40% RH | |
| | Wetting Fluid surface | | |
| | tension | | |
| | Viscosity | 2.2 | mPas |

### Experimental Examples

### Lentivirus Production and Clarification

Lentivirus with green fluorescent protein (GFP) insert was produced in HEK293T suspension cells medium after transfection with four plasmids according to conventional methods. At harvest the feed was treated with 10 U/mL nuclease (Denarase) with 2 mM MgCl₂ and cells were sedimented before clarification using 10 μm, 5 μm, 0.5 μm and 0.2 μm filters. The clarified feed lentivirus titer was ≥E9 VP/mL and ≥E6 TU/mL.

### Capture of Lentivirus Using Weak AIEX Chromatography

The clarified feed was applied to a 5 ml HiTrap™ Capto™ DEAE chromatography column (Cytiva, Sweden) connected to an ÄKTA™ Pure 25 (Cytiva, Sweden) equilibrated in 50 mM Tris-HCl, pH 8.0 (buffer A). The protocol for eluting lentivirus using a short linear NaCl gradient ranging from 130 to 650 mM can be seen in Table 2. “CV” denotes column volume. Directly after elution the eluate was diluted 1:5 with 5% sucrose to stabilize the virus and retain infectivity.

Flowrate was 5 ml/min (1 min residence time) for all steps. All runs were performed in duplicates.

TABLE 2

| Phase | Composition | Volume | |
|---|---|---|---|
| Equilibration | 50 mM Tris-HCl pH 8.0, 130 mM NaCl | 3-10 | CV |
| Sample | Lentivirus feed (load ~ E11 VP/mL resin) | 48-120 | mL |
| loading | | | |
| Wash | 50 mM Tris-HCl pH 8.0, 130 mM NaCl | 10 | CV |
| Elution | 50 mM Tris-HCl pH 8.0, 130-650 mM NaCl | 25 | mL |
| Wash | 50 mM Tris-HCl pH 8.0, 1.3M NaCl | 10 | CV |
| CIP | 1M NaOH | 15 | CV |
| Wash | 50 mM Tris-HCl pH 8.0, 1.3M NaCl | 15 | CV |
| Re- | 50 mM Tris-HCl pH 8.0, 130 mM NaCl | 10 | CV |
| equilibration | | | |

Scavenging Impurities Using Layered Bead Resin with HIC Ligand at Different pH and Flow Rates

The eluate from the weak AIEX capture step was applied to a 1 mL HiTrap™ Capto™ core 700 column (Cytiva, Sweden) connected to an ÄKTA™ Pure 25 (Cytiva, Sweden) equilibrated in 50 mM Tris-HCl at pH 8.0, 7.4 or 7.0, respectively. Lentivirus load was approximately E11 VP in 50 mM Tris-HCl at pH 7.0, 7.4 or 8.0, 4% sucrose and 130 mM NaCl. For pH 7.0, flowrates evaluated were 0.3 mL/min, 0.9 ml/min, 1.4 mL/min and 1.9 ml/min (corresponding to 3.3, 1.1, 0.7 and 0.5 min residence time). All runs were performed in duplicates.

Fractions were analyzed for physical/total viral particles (p24 ELISA, VP/mL), Infectious virus (cell-based transduction assay measuring GFP producing cells after transduction, TU/mL), total protein (micro BCA kit, μg/mL) and total DNA (picoGreen assay, ng/ml).

### Results

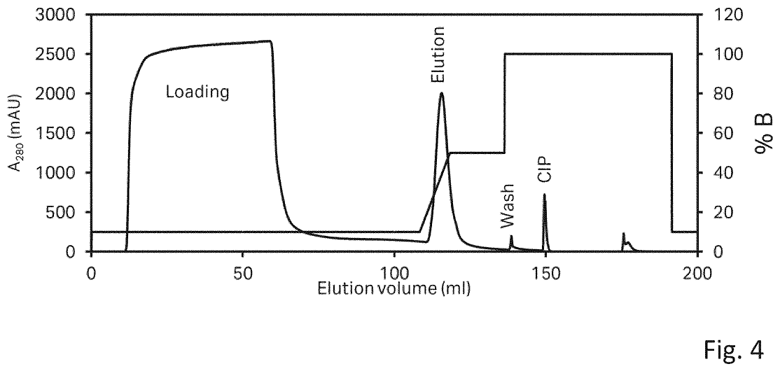

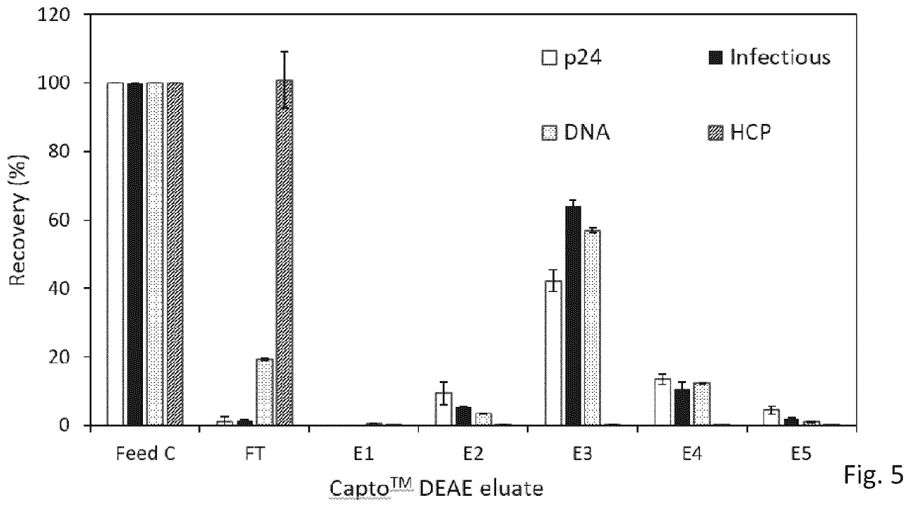

The capture chromatogram using weak anion exchange with a short NaCl gradient elution is shown in FIG. 4. Capture with Capto™ DEAE resulted in >60% infectious lentivirus recovery in the pooled gradient (FIG. 5). The gradient was fractionated into 5 fractions and most of the lentivirus eluted in elution fraction 2 and 3. Most of the host cell protein (HCP) (98% of total protein) was obtained in the flow through and wash, but about 50% of the DNA co-eluted with the lentivirus (FIG. 5).

The Capto™ DEAE eluate was diluted with a buffer of pH 7.0, 7.4 or 8 and applied to a Capto™ core 700 column as described above. The flow through fractions were collected and analyzed. It was found that the buffer with pH of 7.0 resulted in an infectious recovery of 80%, which was a very significant improvement over the recovery rates obtained with buffers of pH 8.0 or 7.4, which were about 35% (FIG. 6). All pH buffers resulted in similar p24 recovery levels. Impurity levels were below limit of detection for both HCP and DNA (BCA, picogreen) for all pH (data not shown).

Lastly, the evaluation if using different flow rates as described above showed that a flow rate of up to 1.4 mL/min, ≥0.7 min residence time, could be used with excellent lentivirus infectious recovery (close to 100%). Also 1.9 mL/min, or 0.5 min residence time, can be used at pH 7.0 with an infectious recovery that is much improved in relation to prior art processes (70%). Impurity levels were below limit of detection for both HCP and DNA (BCA, picogreen) for all flow rates.

### REFERENCES

Kinetic Analyses of Stability of Simple and Complex Retroviral Vectors, F. Higashikawa et al., Virology 280, 124-131 (2001)
Process development of lentiviral vector expression, purification and formulation for gene therapy applications, Doctoral thesis, Sara Nilsson, U C L, 2016
WO 2009/131526 (GE HEALTHCARE BIO SCIENCES AB)

1. A process of purifying enveloped or membranous biological particles from a feed, the process comprising:
a) adding a feed comprising enveloped or membranous biological particles and one or more impurities to a first chromatography medium at a pH of 6-10, preferably 6-8, the first chromatography medium comprising a support material that is functionalized with a ligand that captures said enveloped or membranous biological particles,
b) eluting the enveloped or membranous biological particles from the first chromatography medium in at least one eluate fraction containing enveloped or membranous biological particles,
c) adjusting, if needed, the pH of the eluate fraction of step b) to a pH of less than 7.4,
d) adding the eluate fraction, at a pH in the range of from 6.0 to less than 7.4, to a second chromatography medium comprising porous beads having an inner porous core and an outer porous shell, wherein the core is capable of binding molecules via hydrophobic interactions, and wherein the pore size of the shell prevents particles having a size of 20 nm and larger from contacting the core, and
e) obtaining at least one flow-through fraction containing the purified enveloped or membranous biological particles from the chromatography medium of step d).

2. The process of claim 1, wherein the enveloped or membranous biological particles are selected from enveloped virus particles and exosomes.

3. The process of claim 1 or 2, wherein the ligand that captures said enveloped or membranous biological particles is selected from affinity ligands and anion exchange ligands.

4. The process of claim 2, wherein the support material of the first chromatography medium is functionalized with an anion exchange ligand comprising a diamine functionality generating at least one weak anion exchange group to an ionic capacity of 10-500 μmol/mL.

5. The process of claim 4, wherein the anion exchange group is positively charged or partially positively charged at a pH of 6-10.

6. The process of claim 4, wherein the ligand is described by the formula (I): 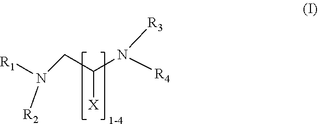
wherein X, for each occurrence independently, is selected from H, OH or a C1-3 group, and
R1, R2, R3 and R4 are independently selected from H, and a C1-3 group,
wherein a C3 group is straight or branched,
wherein a C₁-3 group comprises groups independently selected from OH, O—C1-2, S—C1-2, NH, NHR, NR2,
wherein R is selected from H and a C1-3 group.

7. The process of claim 6, wherein the anion exchange ligand is selected from N,N,N′-triethylethylenediamine, diethylenetriamine, N,N′-dimethylethylenediamine, N-methylethylenediamine, 1,3-diaminopropane, 1,3-diamino-2-hydroxypropane, 2-methyl-1,3-propanediamine and N,N-diethylethylenediamine, and preferably is N,N-diethylethylenediamine.

8. The process of claim 1, wherein the support material of the first chromatography medium is selected from monoliths, membranes, porous beads, non-porous beads, magnetic beads, or expanded bed media.

9. The process of claim 1, wherein the support material of the first chromatography medium is a non-woven fibrous material having a mean flow pore size of 0.1-2.0 μm.

10. The process of claim 1, the ligand that captures said enveloped or membranous biological particles is connected to the support material through an extender group selected from polysaccharide structures and polymeric structures.

11. The process of claim 1, wherein the eluting in step b) is provided using an increasing salt concentration.

12. The process of claim 1, wherein the eluting in step b) is provided by contacting the first chromatography medium with an elution buffer having a salt concentration of at most 0.65 M.

13. The process of claim 1, wherein the pore size of the shell does not allow molecules larger than 700 kDa to contact the core.

14. The process of claim 13, wherein the pore size of the shell does not allow molecules larger than 400 kDa to contact the core.

15. The process of claim 1, wherein the pore size of the shell does not allow particles having a size of 20 nm and above, 30 nm and above, 60 nm and above, 100 nm and above, to contact the core.

16. The process of claim 1, wherein the porous beads comprise a hydrophilic polymer, a polysaccharide, such as agarose.

17. The process of claim 1, wherein the porous core is functionalized with a hydrophobic interaction ligand, preferably wherein the hydrophobic interaction ligand comprises an aliphatic or aromatic C4 to C16 hydrocarbon or an aliphatic C4 to C16 hydrocarbon.

18. The process of claim 17, wherein the hydrophobic interaction ligand is a C4 to C16 alkylamine, or octylamine.

19. The process of claim 1, wherein the eluate fraction is added to the chromatography medium in step d) at a pH of from 6.5 to 7.2, from 6.5 to 7.1, or from 6.8 to 7.1.

20. The process of claim 1, wherein the residence time of the eluate fraction in the chromatography medium of step d) is between 0.5 and 10 minutes.

21. The process of claim 1, wherein the eluate fraction is added to the chromatography medium of step d) at a flow rate of from 0.1 mL/min to 3 mL/min.

22. The process of claim 1, wherein the feed has been subjected to nuclease treatment prior to step a).

23. The process of claim 1, wherein the at least one flow-through fraction containing purified enveloped or membranous biological particles obtained in step e) contains no detectable DNA.

24. Use of a chromatography medium comprising porous beads having an inner porous core and an outer porous shell, wherein the core is capable of binding molecules via hydrophobic interactions, and wherein the pore size of the shell does not allow particles having a size of 20 nm and larger to permeate into the bead and interact with the core, for chromatographic separation of enveloped or membranous biological particles from impurities, or contaminant DNA, at a pH of less than 7.4.

25. The use according to claim 24, wherein the pore size of the shell does not allow molecules having a molecular weight of 400 kDa and above to permeate into the bead and interact with the ligand of the core.

26. The use of claim 24, wherein the pore size of the shell does not allow particles having a size of 20 nm and above, 30 nm and above, 60 nm and above, 100 nm and above, to permeate into the bead and interact with the ligand of the core.

27. The use of claim 24, wherein the core is functionalized with a hydrophobic interaction ligand.
